(19) United States
(12) Patent Application Publication
(10) Pub. No.: US 2009/0076735 A1
(43) Pub. Date: Mar. 19, 2009

(54) Method, system and software arrangement for comparative analysis and phylogeny with whole-genome optical maps

(75) Inventors: Adam Briska, Madison, WI (US); Jacob Schwartz, New York, NY (US); Bing Sun, Jersey City, NJ (US); Bhubaneswar Mishra, Great Neck, NY (US)

Correspondence Address: COOLEY GODWARD KRONISH LLP;ATTN: Patent Group, Suite 1100, 777 - 6th Street, NW, WASHINGTON, DC, 20001, US

(73) Assignees: OpGen, Inc., Madison, WI (US); New York University, New York, NY (US)

(21) Appl. No.: 12/228,870

(22) Filed: Aug. 15, 2008

Related U.S. Application Data
(60) Provisional application No. 60/955,955, filed on Aug. 15, 2007.

(51) Int. Cl. G01N 33/48; G06G 7/48

(52) U.S. Cl. 702/20; 703/11

(57) ABSTRACT

The present invention provides a method for organizing genomic information from multiple organisms. In one embodiment of the invention, phylogenetic trees can be constructed for the organisms. The method of the present invention is termed CAPO, Comparative Analysis and Phylogeny with Optical-Maps. Optical maps of organisms are obtained and phylogeny between the organisms is determined by optical map comparison and bipartite graph matching between the organisms, as, for example, computed by a stable marriage algorithm.

### FIELD OF THE INVENTION

The present invention relates generally to methods, systems and software arrangements for characterizing whole genomes of several species and strains by comparing and organizing their genomes in a searchable database.

### BACKGROUND

A phylogenetic tree represents the evolutionary history among organisms. Constructing phylogenetic trees is a crucial step for biologists to find out how today's extant species are related to one another in terms of common ancestors. Numerous computer tools have been developed to construct such trees

Given DNA sequences of various taxa, the standard technique in evolutionary analysis is to first perform a multiple sequence alignment (on DNA sequences or protein sequences). From the resultant distance matrix, a phylogenetic tree is built describing the relationship of the various taxa with respect to one another. These distance-based methods compress sequence information into a single number and the two sequences with shortest distance are considered as closely related taxa. However, the high cost of sequencing techniques and the biological diversity among the genomes, make it impossible to study phylogeny using detailed sequences of many strains of large-number of related species.

Standard methods for constructing phylogenetic trees, known to persons having ordinary skills in the art, include Unweighted Pair Group Method using Arithmetic Average (P. Sneath and R. Sokal. The principles and practice of numerical classification. Numerical Taxonomy, W. H. Freeman, San Francisco, 1973, incorporated herein by reference), Neighbor Joining (N. Saitou and M. Nei. The neighbor-joining method: a new method for reconstructing phylogenetic trees. Mol. Biol. Evol., 4:406-425, 1987, incorporated herein by reference), Fitch Margoliash (W. Fitch and E. Margoliash. The construction of phylogenetic trees—a generally applicable method utilizing estimates of the mutation distance obtained from cytochrome c sequences. Science, 155:279-284, 1967, incorporated herein by reference), Maximum Parsimony (J. Felsenstein. A likelihood approach to character weighting and what it tells us about parsimony and compatibility. Biological Journal of Linnean Society, 16:183-196, 1981, incorporated herein by reference), and Maximum Likelihood (J. Felsenstein. Evolutionary trees from DNA sequences: A maximum likelihood approach. Journal of Molecular Evolution, 17:368-376, 1981, incorporated herein by reference).

The Unweighted Pair Group Method with Arithmetic Mean (UPGMA) method is a sequential clustering algorithm. It works by constructing distance matrix, amalgamating two Operational Taxonomy Units (OTUs) at each stage and creating a new internal node in the tree at the same time. Whenever two nodes are merged into a new node, it recalculates the distances between the new nodes and other nodes, repeating the process until all OTUs are grouped in a single cluster. It produces a rooted tree containing all the OTUs at the leaves of the tree. It is suitable for constructing phylogenetic tree of taxa with a relatively constant rate of evolution. It has several advantages: The algorithm is simple and fast. Its main disadvantages are: (1) It implicitly assumes the existence of an ultrametric tree: the total branch lengths from the root to any leaf are all equal. In other words, there is an assumed “molecular clock,” which ticks at a constant pace, and all the observed species are at an equal number of ticks from the root; the same evolution rate is assumed to apply to all branches, which is often not the case. (2) It assumes a stringent additive property.

The Neighbor Joining (NJ) method is a heuristic greedy algorithm. It begins with distance matrix and a star-like tree. At each stage two closest neighbors are joined into a new node, which becomes the root of the new tree. The branch lengths from the two nodes to the new node are calculated. The two nodes are replaced by the new node in the distance matrix, thus reducing the number of OTUs by 1. In the process, it updates the distance matrix and performs the node merging process again. The process repeats until there are two OTUs left and they are joined into a root node. Unlike UPGMA, which chooses the neighbors with minimum distance, NJ chooses the neighbors that minimize the sum of branch lengths at each stage. It has several advantages: (1) It is fast and well suited for data sets of substantial size and also for the postprocessing step of bootstrap analysis. (2) It is especially suitable when the rate of evolution of the separate lineages under consideration varies. Its main disadvantages are: (1) It depends heavily on the evolutionary model applied. (2) Like UPGMA, it assumes a stringent additive property.

Both UPGMA and NJ employ distance matrix to reflect evolutionary relationship, compressing sequence information into a single number, and thus cannot reflect the changes of character states of sequences. UPGMA and NJ are relatively fast, so they are suitable for analyzing large data set that is not very strongly similar. In general, NJ gives better result than UPGMA.

The Fitch Margoliash (FM) method assumes that the expected error is proportional to the square root of the observed distances. It compares the two most closely related taxa to the average of all the other taxa. It then moves through the tree sequentially to calculate the distances between decreasingly related taxa until all the distances are found. Its advantages include the following: It does not assume a constant rate of evolution and therefore can produce varied branch lengths from a common ancestor. Its main disadvantage is that it requires longer computational execution time than UPGMA and NJ.

The Maximum Parsimony (MP) method is built upon the principle that simple hypotheses are more preferable than complicated ones. Consequently, the construction of the tree using this method requires the smallest number of evolutionary changes among the OTUs in order to explain the phylogeny of the species under study. This method compares different parsimonious trees and chooses the tree that has the least number of evolutionary steps (substitutions of nucleotides in the context of DNA sequence). MP is a character-based Maximum Parsimony algorithm. It starts with multiple alignment and construct all possible topologies. Based on evolutionary changes, it scores each of these topologies and chooses a tree with the fewest evolutionary changes as the final tree. An evolutionary change is the transformation from one character state to another. Character states can be DNA bases, the loss or gain of a restricted site, and the absence or presence of morphological features. Its advantages are enumerated as follows: (1) It allows the use of all known evolutionary information in tree building. (2) It produces numerous unrooted, “most parsimonious trees.” Some of its disadvantages are listed below: (1) It requires long computation time, although faster than maximum likelihood. (2) It yields little information about branch length. (3) It usually performs well with closely related sequences, but often performs badly with very distantly related sequences.

The Maximum Likelihood (ML) method evaluates the topologies of different trees and chooses the best tree among all as measured with respect to a specified model. Such a model may be based on the evolutionary process that can account for the conversion of one sequence into another. It evaluates a hypothesis about evolutionary history in terms of the probability that the proposed model and the hypothesized history would give rise to the observed data set. The parameter considered in the topology is the branch length. It starts with a multiple alignment and lists all possible topologies of each data partition. It then calculates probability of all possible topologies for each data partition and combines data partitions. It identifies tree with the highest overall probability at all partitions as most likely phylogeny. Its advantages include the following: (1) It is more accurate than other methods. It is often used to test an existing tree. (2) All the sequence information is used. (3) Sampling errors have least effect on the method. Its main disadvantage is that it is extremely slow, and thus impractical for analyzing large data set.

### SUMMARY OF THE INVENTION

The present invention provides a method for organizing genomic information from multiple organisms. In one embodiment of the invention, phylogenetic trees can be constructed for the organisms. The method of the present invention is termed CAPO, Comparative Analysis and Phylogeny with Optical-Maps. This method can be used to determine phylogeny among optical maps of multiple strains or genomes. The low cost and high speed of an Optical Mapping technique provides an elegant solution to the problem posed by the high cost procedures involved in sequence generation and comparison.

In one aspect, the invention provides a method for comparative genomic analysis, the method includes comparing optical maps obtained from one or more organisms in order to obtain at least one pair-wise similarity value; and determining relatedness of the organisms based on said pair-wise similarity value. In a related embodiment, the method further includes constructing a phylogenetic tree based on the relatedness of the organisms. Exemplary organisms include a microorganism, a bacterium, a virus, and a fungus.

Another aspect of the invention provides a method for identifying an unknown organism, the method includes comparing an optical map from an unknown organism to a plurality of optical maps from a phylogenetic tree of known organisms; obtaining a pair-wise similarity value for one or more comparisons between the unknown organism and the known organism in the phylogenetic tree; and identifying the unknown organism based on the pair-wise similarity values. In a related embodiment, the method further includes, prior to the comparing step, preparing an optical map from the unknown organism. In another related embodiment, the method further includes, prior to the comparing step, constructing a phylogenetic tree of known organisms.

Another aspect of the invention provides a method for constructing a phylogenetic tree, the method includes obtaining pair-wise distances among organisms by comparing at least one pair of optical maps from the organisms in order to generate a pair-wise similarity matrix; and constructing a phylogenetic tree based on the pair-wise similarity matrix. In a related embodiment, the method further includes, prior to said obtaining step, preparing optical maps of each organism.

Some of the steps of the methods can be accomplished by a computer utilizing various algorithms. Software instructions to perform embodiments of the invention may be stored on a computer readable medium such as a compact disc (CD), a diskette, a tape, a file, or any other computer readable storage device.

To begin the organization of genomic information, whole-genome physical maps or sequences of multiple organisms are obtained. These maps can either be partially or fully assembled. In one suitable embodiment the physical maps are optical maps. Suitable optical maps include, but are not limited to, restriction enzyme optical maps and probe hybridization optical maps. Once these maps are obtained, the maps of any two organisms are compared.

In one embodiment this comparison is done by using pair-wise map similarity values found by comparing the optical maps of organisms. The distance between the two optical maps (labeled mapA and map B) is found by taking: (alignedL_{A}+alignedL_{B})/(L_{A}+L_{B}), where alignedL_{A} is the length (in units of base pairs, bps) of aligned restriction fragments of mapA, and L_{A} is the total length (also in bps) of restriction fragments of mapA.

After the percentage similarity values are computed, these values are fed into a statistical package available in the language “R” and analyzed with a clustering method, which can be the nearest neighbor, furthest neighbor, or UPGMA

In another embodiment, the distance between the two optical maps is computed by a heuristic mer-based algorithm for pair-wise optical map comparison. After choosing a mer size k, the algorithm is used to generate all k-mers in an optical map for both forward and backward orientations. A k-mer is an optical map segment of length k fragments. For each genome, some k-mers occur much more, or less, frequently than chance predicts (to within a some sizing tolerance), and the distribution of k-mer frequencies comprises a type of “species signatures”. The difference between k-mer distributions and profiles for two species increases as evolutionary distance increases, thus comparing k-mer profiles can be used to infer phylogenetic relationships.

To compare two optical maps i and j, the algorithm examines all common k-mers between them to count the number of common k-mers as cᵢⱼ, and computes the pair-wise map similarity sᵢⱼ, where sᵢⱼ=(sᵢ+sⱼ−2cᵢⱼ)/(sᵢ+sⱼ), where sᵢ and sⱼ are the sizes (all measured in terms of the numbers of restriction fragments) of the two optical maps. sᵢⱼ=0 if i=j. In one embodiment the common mers are computed by accounting for the sizing error. Given two k-mers, k₁=(f₁, f₂, . . . , fₖ) in map 1 and k₂=(g₁, g₂, . . . , gₖ) in map 2 (f's and g's are both measured in units of base pairs, bps), it considers k₁ and k₂ as a pair of common k-mers if and only if the following condition is true:

$$\frac{{F}_{i} ⋂ {G}_{i}}{{F}_{i} ⋃ {G}_{i}} \geq ρ , for all 1 \leq i \leq k .$$

where Fᵢ is interval (fᵢ−σ_{fi}, fᵢ+σ_{fi}), σ_{fi} is the standard deviation for fragment fᵢ; Gᵢ is defined similarly. Threshold ρ is a cutoff determining the least overlap degree between two common intervals, deemed necessary to interpret them as equal modulo statistical noise.

After the pair-wise distances among the organisms are found, a plurality of disjoint pairs of near neighbors among the organisms or their putative ancestors is obtained. In one embodiment a single pair of nearest neighbors is determined by searching all pair-wise possibilities. In another embodiment, multiple pairs of nearest neighbors are determined by using a stable marriage algorithm.

Once the nearest neighbors are determined, the plurality of pairs of neighbors are joined pair-wise to create a set of putative ancestral genomes. The determination of the plurality of disjoint pairs of near neighbors, and the pair-wise joining of such neighbors are repeated until no pair remains. These iterative steps organize the physical maps in a phylogenetic tree.

Another aspect of the invention provides a method for determining similarity among organisms, the method including, comparing optical maps from the organisms to determine relatedness of the organisms.

Other aspects of the invention will become apparent by consideration of the detailed description and accompanying drawings.

### BRIEF DESCRIPTION OF THE DRAWINGS

FIG. 1 is a chart showing the procedure of selecting an appropriate method to infer phylogeny given single-gene sequences.

FIG. 2 shows an example of building a bipartite graph given a distance matrix. A) A distance matrix M of four items (A, B, C, D). B) The corresponding bipartite graph.

FIG. 3 shows a first-degree polynomial fit for restriction fragment sizing error. (a) L vs. StdDev(L), cc=0.7428; (b) √L vs. StdDev(L), cc=0.7562; (c) 1/√L vs. StdDev(L)/L, cc=0.8290.

FIG. 4 shows Data Set I: 11 Escherichia coli Strains.

FIG. 5 shows view maps in Data set I using MapViewer. A pair-wise alignment between Escherichia coli O157:H7 str. Sakai and Escherichia coli O157:H7 EDL933 is shown.

FIG. 6 is a table showing data Set II: 28 Enterobacteriaceae Taxa.

FIG. 7 shows view maps in Data set II using MapViewer

FIG. 8 shows a Phylogenetic tree for data set I and II (k=2, p=0.9)

FIG. 9 shows a Phylogenetic tree for data set I and II (k=3, p=0.8)

FIG. 10 shows a Phylogenetic tree for data set I and II (k=4, p=0.7)

FIG. 11 shows a number of clusters in the iterations of the experiments of data set I and II using CAPO SM-UPGMA/SM-NJ.

FIG. 12 shows Phylogenetic trees constructed by CAPO for data set I and II using default setting and single merge mode.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

### DETAILED DESCRIPTION OF THE INVENTION

A phylogenetic tree represents the evolutionary history among organisms. Some methods have been proposed and implemented for the construction of phylogenetic trees. They can be classified into two groups, the phenetic method (distance matrix method, P. Sneath and R. Sokal. The principles and practice of numerical classification. Numerical Taxonomy, W. H. Freeman, San Francisco, 1973, incorporated herein by reference) and the cladistic methods (maximum parsimony and maximum likelihood, J. Felsenstein. A likelihood approach to character weighting and what it tells us about parsimony and compatibility. Biological Journal of Linnean Society, 16:183-196, 1981, incorporated herein by reference). Popular programs of constructing phylogenetic trees include PHYLP (Available at evolution.genetics.washington.edu/phylip.html; phylogenetic inference package—J Felsenstein) and PAUP (Available at paup.csit.fsu.edu; phylogenetic analysis using parsimony—Sinauer Assoc.).

The phenetic methods use various measures of overall similarity for the ranking of species. They can use any number or type of characters, but the data has to be converted into a numerical value. The organisms are compared to each other for all of the characters and then the similarities are calculated. After this, the organisms are clustered based on the similarities. Such methods place a greater emphasis on the relationships among data sets than the paths they have taken to arrive at their current states. They do not necessarily reflect evolutionary relations.

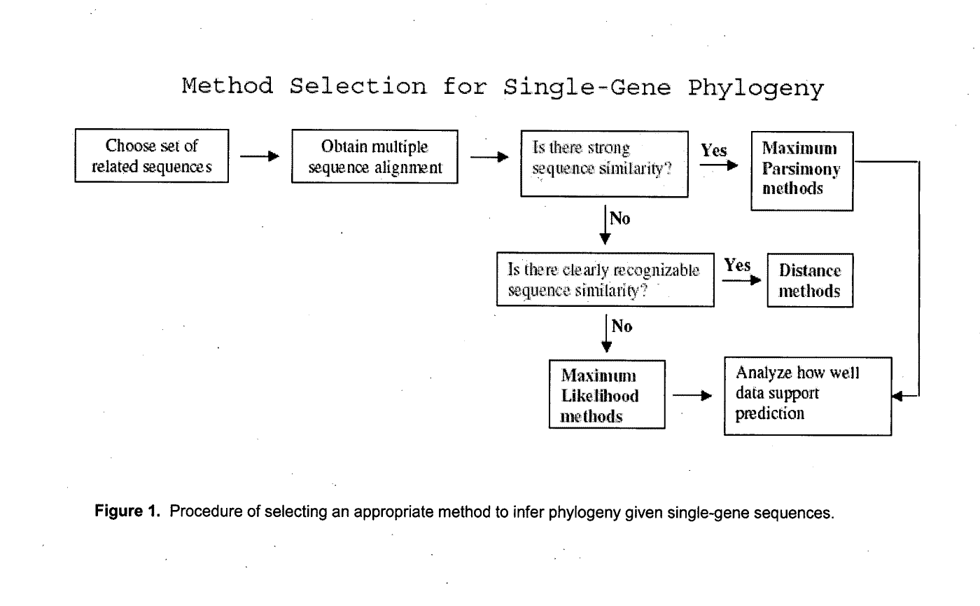

The cladistic method is based on the notion that members of a group share a common evolutionary history and are more closely related to members of the same group than to any other organisms. This method emphasizes the need for large data sets but differs from phenetics in that it does not give equal weight to all characters. Cladists are generally more interested in evolutionary pathways than in relationships. FIG. 1 shows how to select an appropriate method to infer phylogeny given single-gene sequences.

Standard methods for constructing phylogenetic trees, known to persons having ordinary skills in the art, include Unweighted Pair Group Method with Arithmetic Mean (UPGMA), Neighbor Joining (NJ), Fitch Margoliash (FM), Maximum Parsimony (MP), and Maximum Likelihood (ML) methods, and can be combined with certain basic methods related to optical mapping to infer phylogeny using optical-map comparison.

In one embodiment of the present invention, a phylogenetic tree is crafted by using pair-wise map similarity values found by comparing the optical maps of organisms. To calculate the pair-wise map similarity value, a SOMA map aligner is used to find all the local alignments between the two strains above a certain score threshold. Given two optical-maps mapA and mapB, the percentage similarity is found by taking: (alginedL_{A}+alginedL_{B})/(L_{A}+L_{B}), where alginedL_{A} is the length of aligned restriction fragments of mapA, and L_{A} is the total length of restriction fragments of mapA.

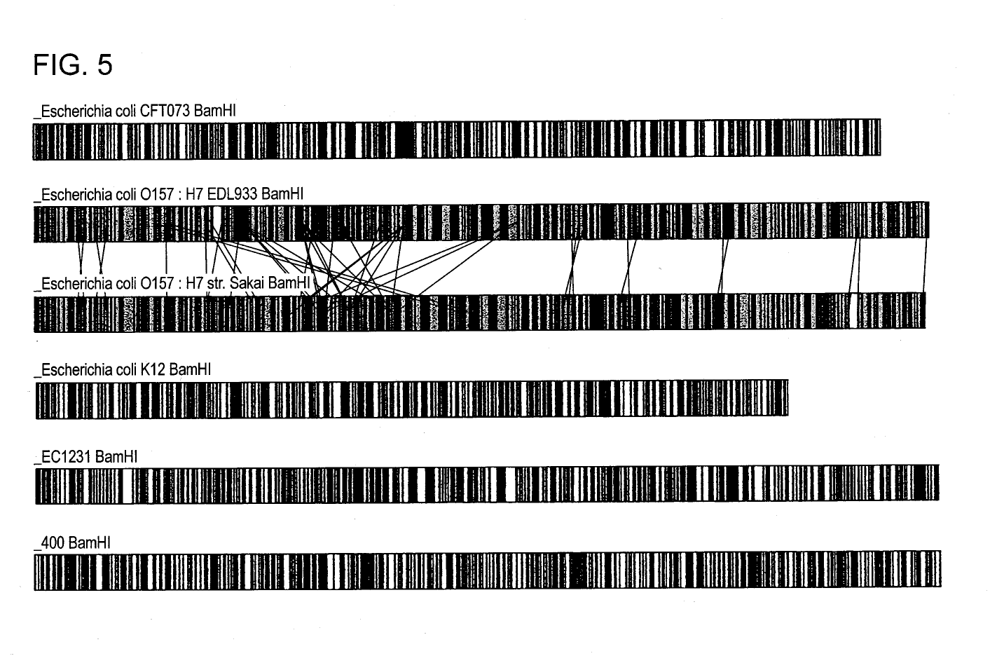

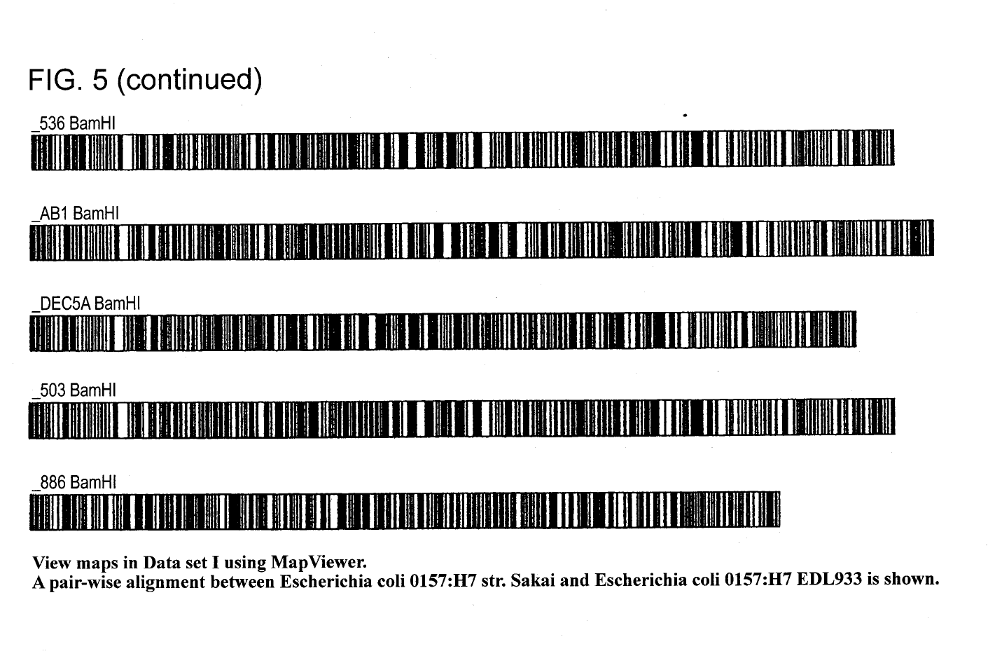

After the percentage similarity values are computed, these values are fed into a statistical package available in the language “R” and analyzed with a clustering method, which can be the nearest neighbor, furthest neighbor, or UPGMA. As an example, a pair-wise alignment was performed between Escherichia coli O157:H7 str. Sakai and Escherichia coli O157:H7 EDL933 using SOMA map aligner with its default settings, shown in FIG. 5.

In another embodiment of the present invention, the distance between the two optical maps is computed by a heuristic mer-based algorithm for pair-wise optical map comparison is used to determine phylogeny among optical maps of multiple strains or genomes.

### Optical Mapping

Optical mapping is a single-molecule technique for production of ordered restriction maps from a single DNA molecule (Samad et al., Genome Res. 5:1-4, 1995). During this method, individual fluorescently labeled DNA molecules are elongated in a flow of agarose between a coverslip and a microscope slide (in the first-generation method) or fixed onto polylysine-treated glass surfaces (in a second-generation method). Id. The added endonuclease cuts the DNA at specific points, and the fragments are imaged. Id. Restriction maps can be constructed based on the number of fragments resulting from the digest. Id. Generally, the final map is an average of fragment sizes derived from similar molecules. Id.

Optical mapping and related methods are described in co-pending U.S. patent application Ser. No. 12/120,586, co-pending U.S. patent application Ser. No. 12/120,592, U.S. Pat. No. 5,405,519, U.S. Pat. No. 5,599,664, U.S. Pat. No. 6,150,089, U.S. Pat. No. 6,147,198, U.S. Pat. No. 5,720,928, U.S. Pat. No. 6,174,671, U.S. Pat. No. 6,294,136, U.S. Pat. No. 6,340,567, U.S. Pat. No. 6,448,012, U.S. Pat. No. 6,509,158, U.S. Pat. No. 6,610,256, and U.S. Pat. No. 6,713,263, each of which is incorporated by reference herein. Optical Maps are constructed as described in Reslewic et al., Appl Environ Microbiol. 2005 September; 71 (9):5511-22, incorporated by reference herein. Briefly, individual chromosomal fragments from test organisms are immobilized on derivatized glass by virtue of electrostatic interactions between the negatively-charged DNA and the positively-charged surface, digested with one or more restriction endonuclease, stained with an intercalating dye such as YOYO-1 (Invitrogen) and positioned onto an automated fluorescent microscope for image analysis. Since the chromosomal fragments are immobilized, the restriction fragments produced by digestion with the restriction endonuclease remain attached to the glass and can be visualized by fluorescence microscopy, after staining with the intercalating dye. The size of each restriction fragment in a chromosomal DNA molecule is measured using image analysis software and identical restriction fragment patterns in different molecules are used to assemble ordered restriction maps covering the entire chromosome.

A current issue with optical map comparison can be understood from the following discussion: An optical map can be viewed as an ordered sequence of “restriction sites,” or equivalently, “restriction fragment lengths.” A vector of decimal numbers, Hₖ=(h₁, h₂, . . . , hₘ), is used to represent a single map k, where hᵢ with index 0<i≦m is the length of the i-th restriction fragment. The size of an optical map k is defined as sₖ=Σhᵢ, hᵢεHₖ. The input to the heuristic mer-based algorithm is an N by M matrix O=(oᵢⱼ), where each row corresponds to an optical map of a strain or a genome. Each column corresponds to a position in that map. N is the total number of maps, and M is the number of restriction fragments in the longest map in that input. Because sequences of different strains or genomes vary in length, the final optical maps usually do not have the same number of restriction fragments. By using the present heuristic mer-based algorithm method, the optical maps are forced to have M fragments by appending zeros to the end of shorter map vectors. Suitably, all the restriction maps in the input must be digested by the same set of restriction endonucleases to make the map comparison meaningful in genome evolution study.

The heuristic mer-based algorithm is based on pair-wise optical map comparison and bipartite graph matching, combined with standard distance methods of phylogeny tree construction. It consists of two major phases. First, pair-wise optical map comparison is performed to generate a pair-wise similarity matrix S=(sᵢⱼ), where sᵢⱼ is the map similarity between the i-th and j-th map in the input matrix O, S is used as input to the second phase of CAPO, which determines phylogeny among input strains or genomes. The output is in the Phylip format, used by many phylogenetic analysis packages. This format consists of a series of nested parentheses describing the branching order with the sequence names. Users can display the phylogeny tree using the NJPLOT program distributed with the ClustalX package (The latest version of the ClustalX program is available at ftp://ftp-igbmc.u-strasbg.fr/pub/ClustalX/). The details of the two algorithms implemented in CAPO are explained in the following sections.

### Pair-Wise Optical Map Comparison

In phase one of constructing a phylogenetic tree, a heuristic mer-based algorithm for pair-wise optical map comparison is used. A ‘mer’ (or more elaborately “restriction-fragment-mer”) in an optical map is an ordered sequence of restriction fragment lengths. A ‘k-mer’ is a mer with k fragment lengths. Mathematically, a k-mer comprises k decimal numbers, and their positions reflect the sequence order of the corresponding restriction fragments. After choosing a mer size k, all k-mers in an optical map for both forward and backward orientations are generated. Each k-mer is indexed by its position in the optical map. To compare two optical maps i and j, all common k-mers between them are examined as follows: the number of common k-mers are counted as cᵢⱼ, and the pair-wise map similarity sᵢⱼ is computed by using the formula sᵢⱼ=(sᵢ+sⱼ−2cᵢⱼ)/(sᵢ+sⱼ), where sᵢ and sⱼ are the sizes of the two optical maps. sᵢⱼ=0 if i=j. The computed pair-wise similarity matrix S is used as input to the next phase of inferring phylogeny.

Common mers are searched in a manner allowing for sizing errors. For example, given two k-mers, k₁=(f₁, f₂, . . . , fₖ) in map 1 and k₂=(g₁, g₂, . . . , gₖ) in map 2, k₁ and k₂ are considered as a pair of common k-mers if and only if the following condition is true:

$$\frac{{F}_{i} ⋂ {G}_{i}}{{F}_{i} ⋃ {G}_{i}} \geq ρ , for all 1 \leq i \leq k . \qquad ( I )$$

where Fᵢ is interval (fᵢ−σf_{fi}, fᵢ+σ_{fi}), σ_{fi} is the standard deviation for fragment fᵢ; Gᵢ is defined similarly. Threshold ρ is a cutoff determining the least overlap degree between two common intervals. The standard deviation of a restriction fragment is estimated via observations of experiment data. Details are given in a later section.

### Inferring Phylogeny

Given a matrix of distances among a set of taxa, both the UPGMA and NJ methods are widely used in phylogenetic analysis to show how similar or dissimilar they are. The UPGMA method assumes equal rates of evolution, so that branch tips come out equal. The NJ method allows for unequal rates of evolution, so that branch lengths are proportional to amount of change. The present method combines the standard stable marriage (SM) algorithm for bipartite graph matching problem with either the UPGMA or the NJ method for inferring phylogeny.

Usually a phylogeny tree is constructed in stepwise manner. Every time two most similar sequences are clustered together, they are combined into a new node, representing their least common ancestor. The clustering process continues until there is only one node left. Therefore, given n taxa, traditional distance-based methods need O(n) iterations to construct a phylogenetic tree. In normal cases, the present method is capable of constructing a phylogenetic tree in log(n) iterations, though its worst-case number of iterations is comparable to traditional distance-based methods. It works as follows:

Initialization: Define T to be the set of leaf nodes, one for each given optical map. If the UPGMA method is used, the distance matrix D=(dᵢⱼ)=(sᵢⱼ), where sᵢⱼ is the map similarity obtained from phase one. If the NJ method is used, uᵢ=Σⱼ₌₁ⁿ sᵢⱼ/(n−2) for each node i in T, where n is the total number of nodes in T. The distance matrix D is recomputed to be D=(dᵢⱼ)=(sᵢⱼ−uᵢ−uⱼ).

Iteration: Build a bipartite graph. Partition D along diagonal line into two parts: the upper triangular part UT and the lower triangular part LT. Pairs in UT form the left-column in the bipartite graph, and pairs in LT form the right column. Each node i has a preference list of nodes, ranked by dᵢⱼ.

Apply the stable marriage algorithm and produce a set X of stable pairs (B. Sun, J. Schwartz, O. Gill, and B. Mishra. Combat: Search rapidly for highly similar protein-coding sequences using bipartite graph matching. In Computational Science-ICCS 2006: 6th International Conf., pages 654-661, Reading, UK., 2006, incorporated herein by reference). Such a ‘stable pair’ is a pair of nodes connected by the stable marriage algorithm and is be clustered into a new internal node if this pair passes the following cleaning step.

Clean the set X: sort stable pairs in decreasing order of dᵢⱼ and keep only the first m pairs in X that are disjoint. Note that two pairs (a, b) and (c, d) are disjoint with each other if and only if no two nodes in different pairs are the same.

Connect nodes and update the distance matrix D in a loop until X is empty. In each loop execute the following operations: I) extract the first pair (i, j) in X; II) join them with a new internal node vᵢⱼ. The node vᵢⱼ has its cluster size nᵢⱼ=nᵢ+ₙⱼ (initially, nᵢ=1).}; III) compute the distances between node vᵢⱼ and the remaining nodes k; IV) delete dᵢⱼ in D and add the new distances to D; V) connect nodes i and j in T with vᵢⱼ.

Termination: When only two nodes i and j remain unconnected in T, connect them to the root node of the tree T.

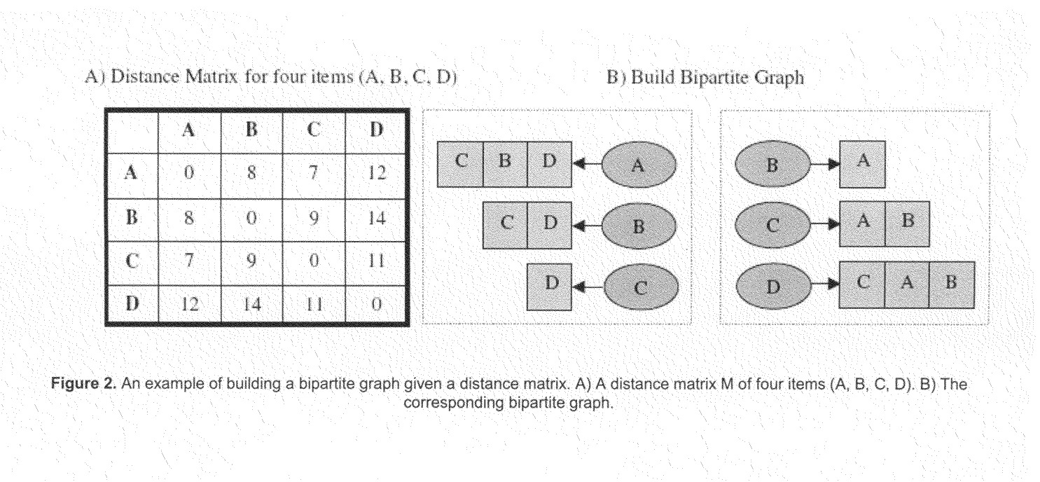

An example of building a bipartite graph given a distance matrix is shown in FIG. 2. Each node has a preference list (gray boxes) ordered by distances. Left panel contains pairs in the upper triangular part of M; right panel contains pairs in the lower triangular part of M. For example, the first row in the left panel means “item A prefers to pair with C, B, D, in the decreasing order of preferences.”

### Correction of Sizing Errors

Optical maps of different strains of the same species would vary due to single nucleotide differences (SNPs), small insertions and deletions (RFLPs) as well as many genomic rearrangement events that leave their footprints on restriction site patterns. Further variations are introduced by the noises in the experimental process. These can be due to: sizing errors, partial digestion, short missing restriction fragments, false cuts, ambiguities in the orientation, optical chimerisms, and so on (T. Anantharaman, B. Mishra, and D. Schwartz. Genomics via optical mapping II: Ordered restriction maps. Journal of Computational Biology, 4(2):91-118, 1997; B. Mishra. Optical mapping. Encyclopedia of the Human Genome, Nature Publishing Group, Macmillan Publishers Limited, London, UK, 4:448-453, 2003, incorporated by reference). These error factors introduced by the experimental process are classified into three types—sizing errors, digestion errors, and orientation errors.

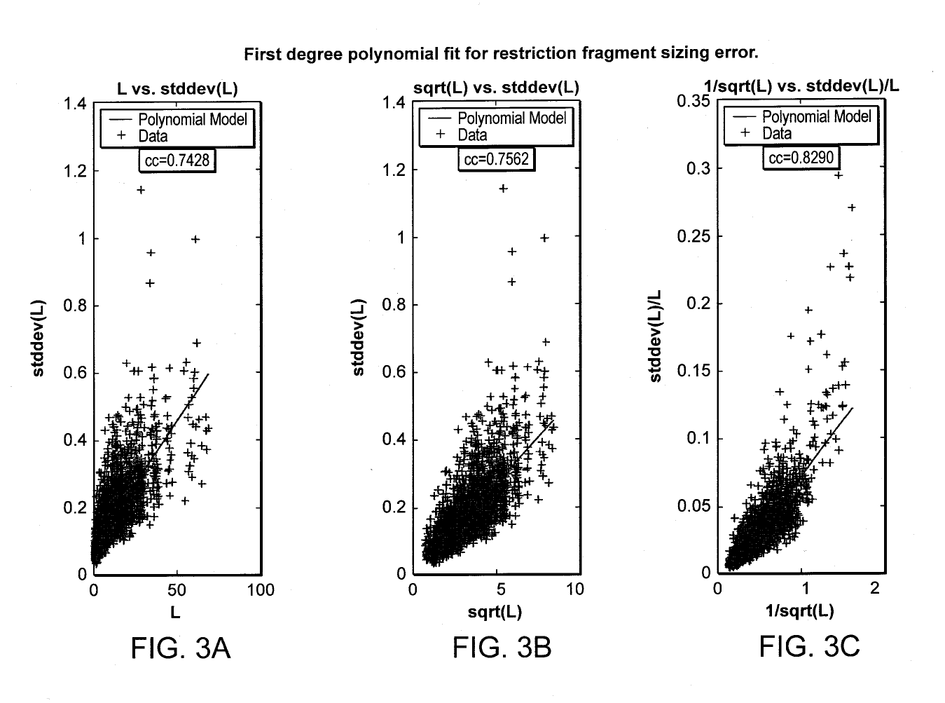

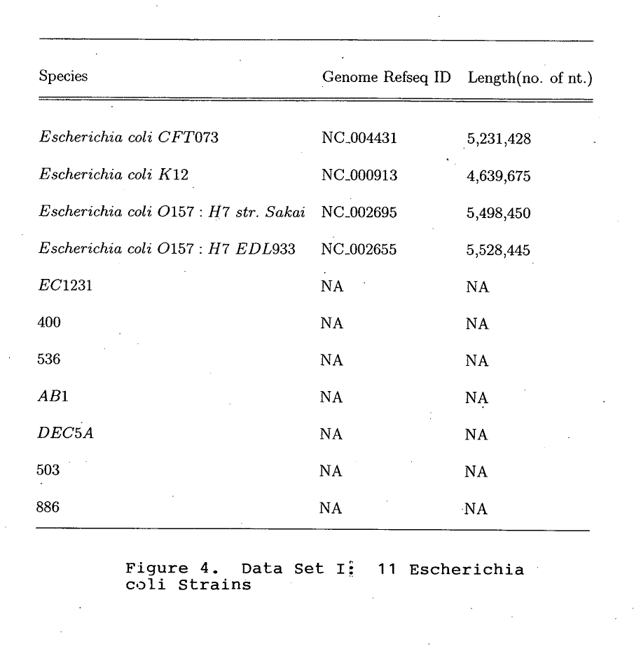

The sizing error statistics is estimated from observations of experiments done by OpGen, Inc. and NYU Bioinformatics Group. These observations (including fragment lengths and standard deviations) are what are reported in the output from the GENTIG (T. Anantharaman, B. Mishra, and D. Schwartz. Genomics via optical mapping III: Contiging genomic DNA and variations; B. Mishra. Optical mapping. Encyclopedia of the Human Genome, Nature Publishing Group, Macmillan Publishers Limited, London, UK, 4:448-453, 2003, incorporated herein by reference) software that OpGen and other practitioners of optical mapping have used to produces optical maps. A first-degree polynomial fit for the three pairs of variables: L˜StdDev(L), √(L)˜StdDev(L), and 1/√(L)˜StdDev(L)/L is shown in FIG. 3, where linear correlation coefficient is referred to as cc. No apparent linear relation is observed between any pair of them since none of these pairs have linear correlation coefficient close enough to one (e.g., >0.95). These results indicate that it may not be appropriate to estimate standard deviations using any of these ‘linear relations.’ Therefore data interpolation is used instead to estimate standard deviations StdDev(L) for a restriction fragment whose length is L. This data interpolation step is performed in the following way: given a fragment length L, find L₁ and Lᵣ from the error plot shown in Figure below (a) where L₁ and Lᵣ are the closest left neighbor and right neighbor of L, respectively (L₁<L<Lᵣ); compute StdDev(L) using StdDev(L)=(StdDev(L₁)+StdDev(Lᵣ))/2.

The invention having now been described, it is further illustrated by the following examples and claims, which are illustrative and are not meant to be further limiting. Those skilled in the art will recognize or be able to ascertain using no more than routine experimentation, numerous equivalents to the specific procedures described herein. Such equivalents are within the scope of the present invention and claims.

The contents of all references and citations, including issued patents, published patent applications, and journal articles cited throughout this application, are hereby incorporated by reference in their entireties for all purposes.

### EXAMPLES

### Creation of Data Set I

Eleven optical maps constructed commercially by OpGen (Website of OpGen Inc. is http://www.opgen.com/) for varying E. coli strains. Information describing this data set is listed in FIG. 4. All the organisms described in data set I are E. coli bacteria, and are identified by their individual strain names. Sequence data is not available for most but four of these E. coli strains, including Escherichia coli CFT073, Escherichia coli K12, Escherichia coli O157:H7 str. Sakai, and Escherichia coli O157:H7 EDL933.

The following procedure was used to produce this data: i) purified chromosomal DNA is deposited onto an optical mapping surface using a microfluidic device; ii) the DNA is encased in a thin layer of acrylamide and incubated with the restriction enzyme BamHI (it cleaves at every site containing the 6 bp long sequence GGATCC) in a humidified chamber at 37° C. for 60˜120 mins; iii) the digested DNA is labeled with fluorescent YOYO-1 and the individual molecules are imaged with fluorescence microscopy; iv) digital images are collected by an automated image-acquisition system and image files are processed to create single-molecule optical maps; v) individual molecule restriction maps are overlapped by using GENTIG (GENomic conTIG) map-assembly software.

Briefly, GENTIG works by comparing single-molecule restriction maps and estimating the probability that these two molecules arose from overlapping genomic locations, where the probability is computed conditional to the likelihood of possible experimental errors resulting from incomplete digestion, spurious cuts, and sizing errors. Through repeated overlapping of molecules, the assembler reconstructs the ordered restriction map of the genome. This technique has been previously applied to map many other bacterial genomes.

A commercially available interface for viewing optical-maps, called MapViewer (available from OpGen, Inc.) is then used. MapViewer allows users to visualize optical-maps, to move maps around, pull up sequence information when available, and change the orientation of the maps. FIG. 5 shows the optical maps for data set I using MapViewer. A pair-wise alignment between Escherichia coli O157:H7 str. Sakai and Escherichia coli O157:H7 EDL933 is shown. Regions that match exactly once are colored green, and regions that match to more than one location are colored red.

Creation of Data Set II

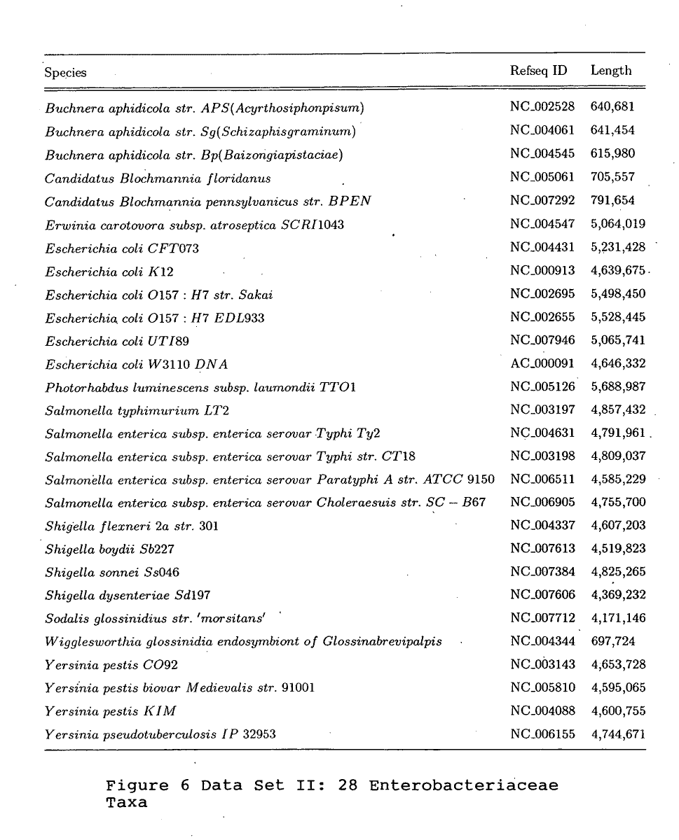

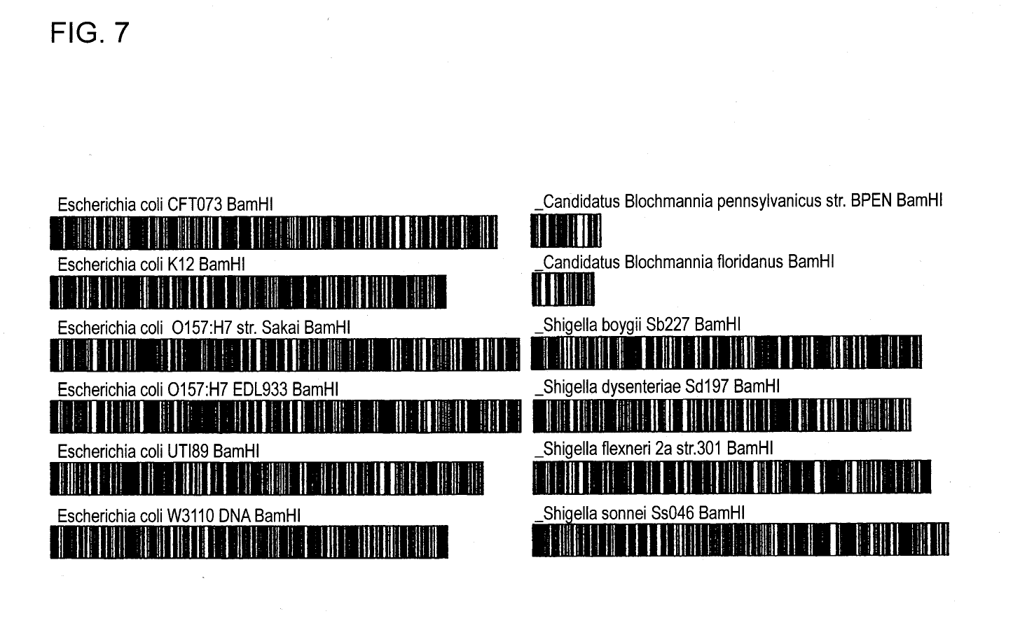

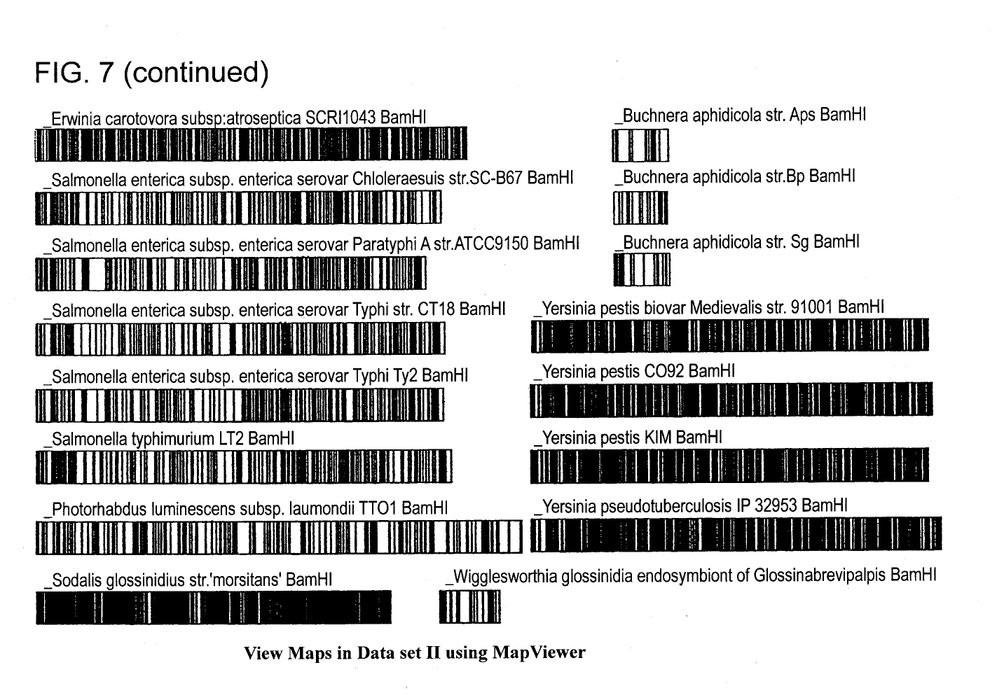

Twenty-eight genomic sequences of Enterobacteriaceae taxa are downloaded from the NCBI database, and then cleaved “in silico” with the restriction enzyme BamHI. Their optical maps were constructed using the SilicoMap software provided by OpGen; The SilicoMap tool is built upon the BioPerl toolkit which is able to perform an in silico restriction digest, after which, it is straightforward to find the lengths of each of the resulting fragments and create the map. Information describing this data set is listed in FIG. 6. FIG. 7 shows the optical maps for data set I using MapViewer.

Analysis of Data Sets

Experimental results are provided in this section using CAPO on both real optical mapping data of eleven E. coli strains and simulated optical mapping data of twenty-eight entire genomes of Enterobacteriaceae taxa. All of the tests were run on a 2.4-GHz Pentium IV machine with 3 GB of RAM.

Parameter Settings

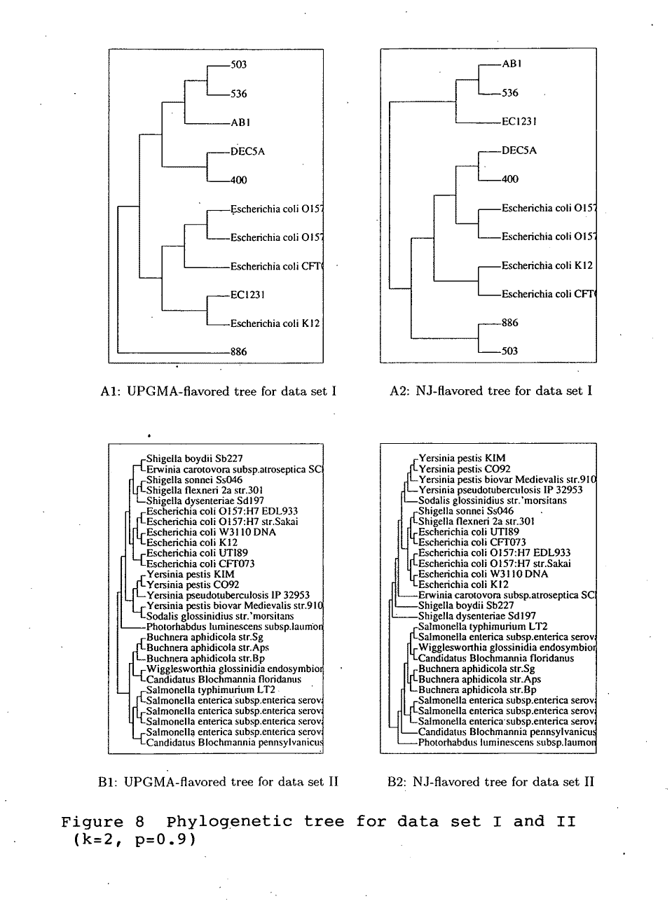

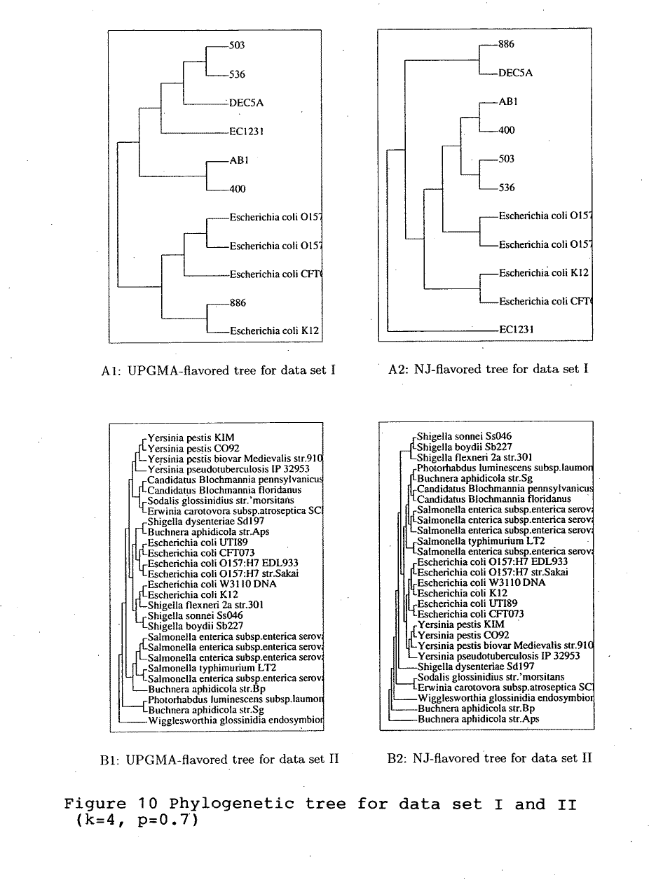

Users have choices for two parameters in CAPO: k (mersize) and p (cutoff value involved in determining whether two restriction fragment lengths are ‘equal’ considering sizing errors). The effect of parameter settings in CAPO is tested in the following experiments using the two data sets: k=2, ρ=0.9 (see FIG. 6), k=3, ρ=0.8 (see FIG. 7) k=4, ρ=0.7 (see FIG. 8). To adequately tolerate sizing errors it was found reasonable to use smaller cutoff value of ρ if a larger mer-size is chosen. Shown in FIG. 8-FIG. 10, the ‘best’ results (whose phylogenetic trees are most biologically meaningful) are produced using k=3, ρ=0.8. k=3, ρ=0.8 was, therefore, subsequently used as the default parameter setting.

Phylogenetic Tree Evaluation

Since there are no ‘true’ phylogenetic trees available for comparison with the results computed by the present method, the quality of these trees were evaluated based on optical map alignments, the taxonomy information given by the NCBI database, and tree topology overlap between the two different distance methods. Using the SOMA map aligner developed by OpGen, it was found that the map of Escherichia coli K12 is very similar to that of 886, and these two strains are clustered closely by the present method with default setting (see FIG. 7, A1, A2). The present method also assigns the rest of three known E. coli strains close evolutionary distances. Using data set II, it was observed that the present method often clustered biologically closely related taxa together (the Buchnera aphidicola strains, the Candidatus Blochmannia strains, the E. coli strains, the Salmonella strains, etc.), as would be desired. Lastly, phylogenetic trees produced by the present method for the same data set using different distance methods were also found to share substantial tree topology overlap.

Cluster Sizes

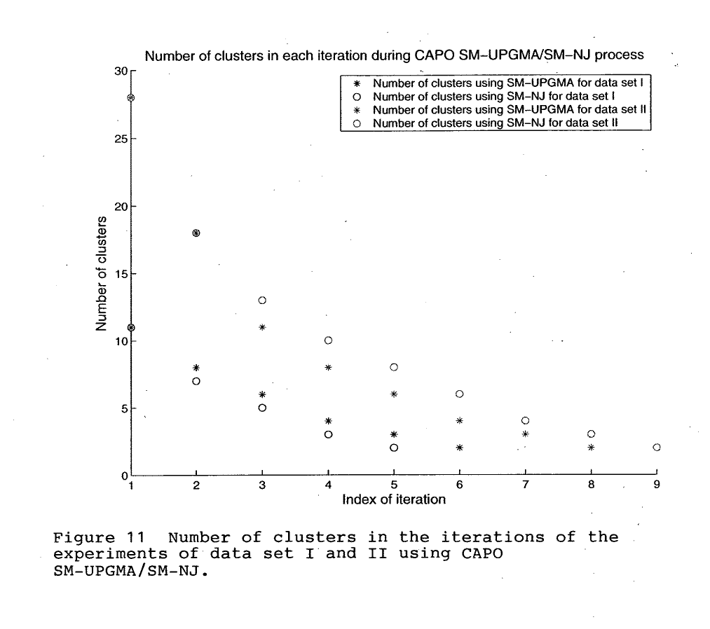

The present method (CAPO) constructs phylogenetic trees in far fewer iterations than standard distance methods. For data set I, CAPO UPGMA-flavored trees and NJ-flavored trees were constructed in 5 and 6 iterations, respectively. For data set II, CAPO UPGMA-flavored trees and NJ-flavored trees were constructed in 8 and 9 iterations, respectively. Number of remaining clusters in each iteration is shown in FIG. 11.

Impact of Single-Merge Mode and Multi-Merge Mode

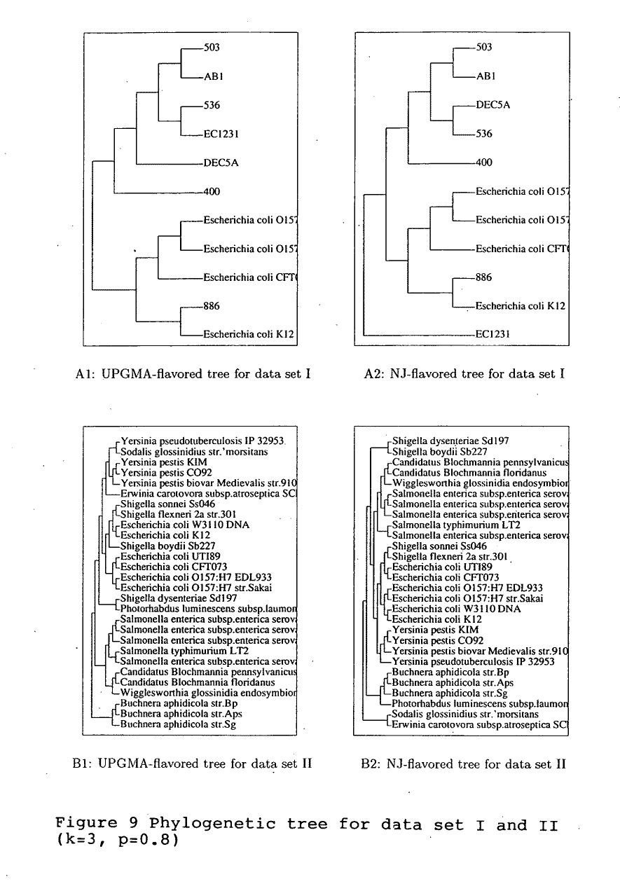

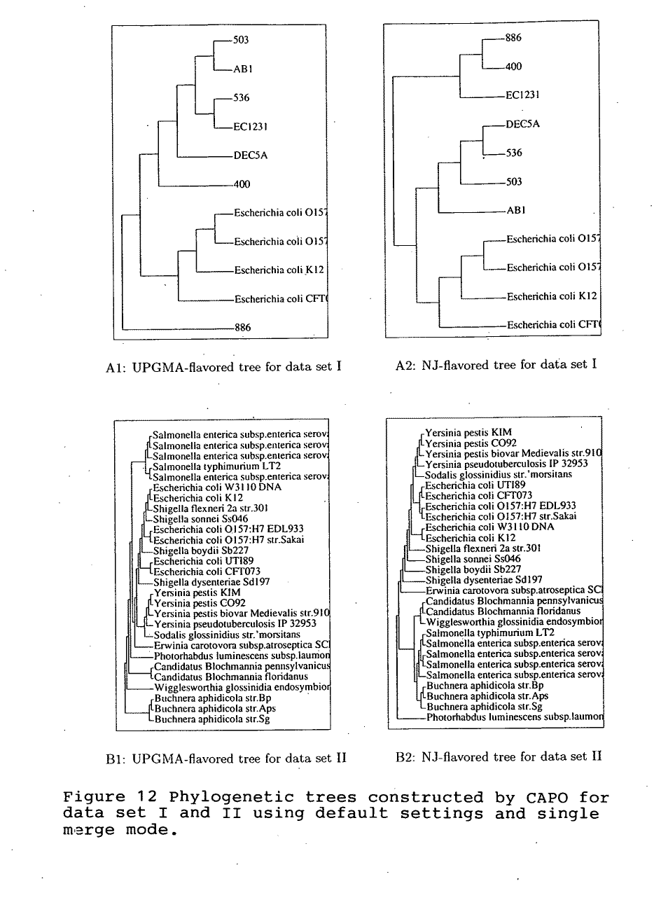

To see if there was any effect on the phylogenetic tree topology by merging more than two clusters in a single iteration. Phylogenetic trees were generated for both data sets using ‘single-merge mode’ (merge exactly two clusters at one iteration), as shown in FIG. 12. Compared with trees produced in ‘multi-merge mode’ (merge multiple pairs of disjoint clusters found by the stable marriage procedure in a single iteration), as shown in FIG. 9, some tree topology changes are shown, especially between FIG. 12-A2 and FIG. 9-A2. Because there is no reliable method for detecting the similarity level between two trees and because there is no prior knowledge about the ‘true’ tree topology, at this point, it remains unclear what the impact of various merging mode could be. However, almost all corresponding trees share substantial tree topology overlap, thus indicating a strong measure of consistency that can be achieved by the present method.

Implementation and Speed

The methods of the present invention are implemented in C++ and all experiments were performed on a Pentium IV PC with 3 GB memory. Experiments for data set I and II took ˜4 sec. and ˜18 sec., respectively. The computational efficiency of CAPO indicates its potential widespread usage in analyzing large genomic data sets.

### BACKGROUND REFERENCES

S. Altschul, T. Madden, A. Schaffer, J. Zhang, Z. Zhang, W. Miller, and D. Lipman. Gapped blast and psi-blastla new generation of protein database search programs. Nucleic Acids Res., 25:3389-3402, 1997.
T. Anantharaman, B. Mishra, and D. Schwartz. Genomics via optical mapping III: Contiging genomic DNA and variations.
T. Anantharaman, B. Mishra, and D. Schwartz. Genomics via optical mapping II: Ordered restriction maps. Journal of Computational Biology, 4(2):91-118, 1997.
T. Anantharaman, V. Mysore, and B. Mishra. Fast and cheap genome wide haplotype construction via optical mapping. volume 10, pages 385-396. Pacific Symposium on Biocomputing, 2005.
C. Aston, B. Mishra, and D. Schwartz. Optical mapping and its potential for large-scale sequencing projects. Trends in Biotechnology, 17:297-302, 1999.
S. Batzoglou, L. Pachter, J. Mesirov, B. Berger, and E. Lander. Human and mouse gene structure: Comparative analysis and application to exon prediction. Genome Res., 10:950-958, 2000.
E. Birney and R. Durbin. Using genewise in the drosophila annotation experiment. Genome Res., 10:547-548, 2000.
E. Birney and et al. Ensembl. Nucleic Acids Res., 32:468-470, 2004.
N. Bray, I. Dubchak, and L. Pachter. Avid: A global alignment program. Genome Res., 13:97-102, 2003.
M. Brudno and B. Morgenstern. Fast and sensitive alignment of large genomic sequences. In Proc. of the IEEE Computer Society Bioinformatics Conference, pages 138-150, 2002.
C. Burge and S. Karlin. Prediction of complete gene structures in human genomic dna. J. Mol. Bio., 268:78-94, 1997.
W. Cai, J. Jing, B. Irvin, L. Ohler, E. Rose, H. Shizuya, U. Kim, M. Simon, T. Anantharaman, B. Mishra, and D. Schwartz. High-resolution restriction maps of bacterial artificial chromosomes constructed by optical mapping. Proc. Natl. Acad. Sci. U.S.A., 95:3390-3395, 1998.
A. Delcher, S. Kasif, R. Fleischmann, J. Peterson, O. White, and S. Salzberg. Alignment of whole genomes. Nucleic Acids Res., 27:2369-2376, 1999.
A. Delcher, A. Phillippy, J. Carlton, and S. Salzberg. Fast algorithms for large-scale genome alignment and comparison. Nucleic Acids Res., 30(11):2478-2483, 2002.
J. Deogun, J. Yang, and F. Ma. Emagen: An efficient approach to multiple whole genome alignment. In the 2nd Asia Pacific Bioinformatics Conference (APBC2004), volume 29, Dunedin, New Zealand, 2004.
J. Felsenstein. Alternative methods of phylogenetic inference and their interrelationship. Systematic Zoology, 28:49-62, 1979.
J. Felsenstein. Evolutionary trees from DNA sequences: A maximum likelihood approach. Journal of Molecular Evolution, 17:368-376, 1981.
J. Felsenstein. A likelihood approach to character weighting and what it tells us about parsimony and compatibility. Biological Journal of Linnean Society, 16:183-196, 1981.
W. Fitch and E. Margoliash. The construction of phylogenetic trees—a generally applicable method utilizing estimates of the mutation distance obtained from cytochrome c sequences. Science, 155:279-284, 1967.
K. Frazer, L. Elnitski, D. Church, I. Dubchak, and R. Hardison. Cross-species sequence comparisons: A review of methods and available resources. Genome Res., 13:1-12, 2003.
D. Gale and L. Shapley. College admissions and the stability of marriage. Am. Math. Monthly, 60(1):9-15, 1962.
M. Gelfand, A. Mironov, and P. Pevzner. Gene recognition via spliced sequence alignment. volume 93, pages 9061-9066, 1996.
A. Goldberg, S. Plotkin, D. Shmoys, and E. Tardos. Using interiorpoint methods for fast parallel algorithms for bipartite matchings and related problems. SIAM Journal on Computing, 21(1):140-150, 1992.
D. Gusfield. Algorithms on Strings, Trees and Sequences: Computer Science and Computational Biology. Cambridge University Press, New York, 1997.
S. Henikoff and J. Henikoff. Amino acid substitution matrices from protein blocks. Proc. Natl. Acad. Sci. USA, 89:10915-10919, 1992.
M. Hohl and E. Ohlebusch. Efficient multiple genome alignment. In Proceedings of the 10th Intervational Conference on Intelligent Systems for Molecular Biology, pages 312-320, 2002.
K. Iwama, D. Manlove, S. Miyazaki, and Y. Morita. Stable marriage with incomplete lists and ties. In Proc. ICALP '99, pages 443-452. 1999.
W. James Kent. Blat—the blast-like alignment tool. Genome Res., 12:656-664, 2002.
J. Jing, Z. Lai, C. Aston, J. Lin, D. Carucci, M. Gardner, B. Mishra, T. Anantharaman, H. Tettelin, L. Cummings, S. Hoffman, J. Venter, and D. Schwartz. Optical mapping of plasmodium falciparum chromosome 2. Genome Res., 9:175-181, 1999.
W. Kent and A. Zahler. Conservation, regulation, synteny, and introns in a large-scale c. briggsae-c. elegans genomic alignment. Genome Res., 10:1115-1125, 2000.
A. Krogh. Using database matches with for hmmgene for automated gene detection in drosophila. Genome Res., 11:817-832, 2000.
M. Kuhner and F. J. A simulation comparison of phylogeny algorithms under equal and unequal evolutionary rates. Mol. Biol. Evol., 11(3):459-468, 1994.
Z. Lai, J. Jing, C. Aston, V. Clarke, J. Apodaca, E. Dimalanta, D. Carucci, M. Gardner, B. Mishra, and et al. A shotgun optical map of the entire plasmodium falciparum genome. Nat. Genet., 23:309-313, 1999.
I. Lee, D. Westaway, A. Smit, K. Wang, J. Seto, L. Chen, C. Acharya, M. Ankener, D. Baskin, C. Cooper, and et al. Complete genomic sequence and analysis of the prion protein gene region from three mammalian species. Genome Res., 8:1022-1037, 1998.
A. Lim, E. Dimalanta, K. Potamousis, G. Yen, J. Apodoca, C. Tao, J. Lin, R. Qi, J. Shiadas, and et al. Shotgun optical maps of the whole Escherichia coli o157:h7 genome. Genome Res., 11:1584-1593, 2001.
J. Lin, R. Qi, C. Aston, J. Jing, T. Anantharaman, B. Mishra, O. White, M. Daly, K. W. Minton, J. Venter, and D. Schwartz. Whole-genome shot-gun optical mapping of deinococcus radiodurans. SCIENCE, 285:1558-1562, 1999.
B. M., C. Do, G. Cooper, M. Kim, and E. Davydov. Lagan and multi-lagan: Efficient tools for large-scale multiple alignment of genomic DNA. Genome Res., 13:721-731, 2003.
E. McCreight. A space-economical suffix tree construction algorithm. J. ACM., 23:262-272, 1976.
S. Melnik, H. Garcia-Molina, and E. Rahm. Similarity Flooding: A versatile graph matching algorithm and its application to schema matching. In Proc. 18th Intl. Conf. on Data Engineering (ICDE), San Jose Calif., 2002.
B. Mishra. Optical mapping. Encyclopedia of the Human Genome, Nature Publishing Group, Macmillan Publishers Limited, London, UK, 4:448-453, 2003.
B. Morgenstern. Dialign 2: improvement of the segment-to-segment approach to multiple sequence alignment. Bioinformatics, 15(3):211-218, 1999.
B. Morgenstern, O. Rinner, S. AbdeddaÄlm, D. Haase, K. Mayer, A. Dress, and H. Mewes. Exon discovery by genomic sequence alignment. Bioinformatics, 18(6):777-787, 2002.
C. Notredame, D. Higgins, and J. Heringa. T-coffee: A novel method for fast and accurate multiple sequence alignment. J. Mol. Biol., 302:205-217, 2000.
H. S. and H. J. G. Performance evaluation of amino acid substitution matrices. Proteins, 17(1):49-61, 1993.
N. Saitou and M. Nei. The neighbor-joining method: a new method for reconstructing phylogenetic trees. Mol. Biol. Evol., 4:406-425, 1987.
S. Schwartz, L. Elnitski, M. Li, M. Weirauch, and et al. Multipipmaker and supporting tools: alignments and analysis of multiple genomic DNA sequences. Nucleic Acids Research, 31(13):3518-3524, 2003.
S. Schwartz, W. Kent, A. Smit, Z. Zhang, R. Baertsch, R. Hardison, D. Haussler, and W. Miller. Human-mouse alignments with blastz. Genome Res., 13:103-107, 2003.
S. Schwartz, Z. Zhang, K. Frazer, A. Smit, C. Riemer, J. Bouck, R. Gibbs, R. Hardison, and W. Miller. Pipmaker-a web server for aligning two genomic DNA sequences. Genome Res., 10:577-586, 2000.
P. Sneath and R. Sokal. The principles and practice of numerical classification. Numerical Taxonomy, W. H. Freeman, San Francisco, 1973.
J. Stajich, D. Block, K. Boulez, S. Brenner, S. Chervitz, C. Dagdigian, and et al. The bioperl toolkit: Perl modules for the life sciences. Genome Res., 12(10):1611-1618, 2002.
B. Sun, J. Schwartz, O. Gill, and B. Mishra. Combat: Search rapidly for highly similar protein-coding sequences using bipartite graph matching. In Computational Science —ICCS 2006: 6th International Conf, pages 654-661, Reading, UK., 2006.
W. Taylor. Protein structure comparison using bipartite graph matching and its application to protein structure classification. Mol. Cell. Proteomics, 1(4):334-339, 2002.
J. Thompson, D. Higgins, and T. Gibson. Clustal w: improving the sensitivity of progressive multiple sequence alignment through sequence weighting, position-specific gap penalties and weight matrix choice. Nucleic Acids Research, 22(22):4673-4680, 1994.

1. A method for comparative genomic analysis, the method comprising:
comparing optical maps obtained from one or more organisms in order to obtain at least one pair-wise similarity value; and
determining relatedness of the organisms based on said pair-wise similarity value.

2. The method according to claim 1, further comprising constructing a phylogenetic tree based on said relatedness of the organisms.

3. The method according to claim 1, wherein the organisms are selected from the group consisting of a microorganism, a bacterium, a virus, and a fungus.

4. A method for identifying an unknown organism, the method comprising:
comparing an optical map from an unknown organism to a plurality of optical maps from a phylogenetic tree of known organisms;
obtaining a pair-wise similarity value for one or more comparisons between the unknown organism and the known organism in the phylogenetic tree; and
identifying the unknown organism based on the pair-wise similarity values.

5. The method according to claim 4, wherein prior to said comparing step, the method further comprises preparing an optical map from the unknown organism.

6. The method according to claim 5, wherein prior to said comparing step, the method further comprises constructing a phylogenetic tree of known organisms.

7. The method according to claim 4, wherein the unknown organism is selected from the group consisting of a microorganism, a bacterium, a virus, and a fungus.

8. A method for constructing a phylogenetic tree, the method comprising:
obtaining pair-wise distances among organisms by comparing at least one pair of optical maps from the organisms in order to generate a pair-wise similarity matrix; and
constructing a phylogenetic tree based on the pair-wise similarity matrix.

9. The method according to claim 8, wherein prior to said obtaining step, the method further comprises preparing optical maps of each organism.

10. The method according to claim 9, wherein the optical maps are ordered restriction enzyme optical maps.

11. The method according to claim 9, wherein the optical maps are probe-hybridized optical maps.

12. The method according to claim 8, wherein the pair-wise distances are computed by:
(alignedL_{A}+alignedL_{B})/(L_{A}+L_{B}), where alignedL_{A} is the length of aligned restriction fragments of a map of a first organism, L_{A} is the total length of restriction fragments of a first organism, alignedL_{B} is the length of aligned restriction fragments of a map of a second organism, and L_{B} is the total length of restriction fragments of the second organism.

13. The method according to claim 8, wherein the pair-wise distances are computed by:
choosing a mer size k, and generating k-mers in the optical maps for both forward and backward orientations;
comparing two optical maps by examining common k-mers between the two optical maps and counting number of common k-mers as cᵢⱼ,
computing the pair-wise distance as similarity sᵢⱼ using the formula sᵢⱼ=(sᵢ+sⱼ−2cᵢⱼ)/(sᵢ+sⱼ), where sᵢ is size of the first optical map and sⱼ is size of the second optical map.

14. The method according to claim 13, wherein the common mers are computed by accounting for the sizing error as follows:
a k-mer in the first map is k₁=(f₁, f₂, . . . , fₖ) and a k-mer in a second map is k₂=(g₁, g₂, . . . , gₖ), and the pair is considered a common k-mer if the following condition is true:
$$\frac{{F}_{i} ⋂ {G}_{i}}{{F}_{i} ⋃ {G}_{i}} \geq ρ , for all 1 \leq i \leq k .$$
where Fᵢ is interval (fᵢ−σ_{fi}, fᵢ+σ_{fi}), σ_{fi} is the standard deviation for fragment fᵢ; Gᵢ is interval (gᵢ−σ_{gi}, gᵢ+σ_{gi}), σ_{gi}; σ_{gi} is the standard deviation for fragment gᵢ; and threshold ρ is a cutoff determining the least overlap degree between two common intervals.

15. The method according to claim 8, wherein said constructing step comprises, (a) obtaining a plurality of disjoint pairs of near neighbors among the organisms or putative ancestors of the organisms, (b) joining pair-wise the previously computed plurality of pairs of neighbors to generate a set of putative ancestral genomes, and repeating steps (a) and (b) until no pairs remain.

16. The method according to claim 15, wherein a single disjoint pair of nearest neighbor is determined by searching all pair-wise possibilities.

17. The method according to claim 15, wherein multiple disjoint pairs of nearest neighbors are determined by using a stable marriage algorithm.

18. The method according to claim 15, wherein a single disjoint pair of nearest neighbors are joined in a single-merge mode.

29. The method according to claim 15, wherein multiple disjoint pairs of nearest neighbors are joined in a multi-merge mode.

20. A method for determining similarity among organisms, the method comprising, comparing optical maps from the organisms to determine relatedness of the organisms.

21. A computer program product for comparative genomic analysis, the computer program product being embodied in a computer readable medium and comprising computer instructions to be executed by a processor for: comparing optical maps obtained from one or more organisms in order to obtain at least one pair-wise similarity value; and determining relatedness of the organisms based on said pair-wise similarity value.

22. A computer program product for identifying an unknown organism, the computer program product being embodied in a computer readable medium and comprising computer instructions to be executed by a processor for: comparing an optical map from an unknown organism to a plurality of optical maps from a phylogenetic tree of known organisms; obtaining a pair-wise similarity value for one or more comparisons between the unknown organism and the known organism in the phylogenetic tree; and identifying the unknown organism based on the pair-wise similarity values.

23. A computer program product for constructing a phylogenetic tree, the computer program product being embodied in a computer readable medium and comprising computer instructions to be executed by a processor for: obtaining pair-wise distances among organisms by comparing at least one pair of optical maps from the organisms in order to generate a pair-wise similarity matrix; and constructing a phylogenetic tree based on the pair-wise similarity matrix.
